(19) United States
(12) Patent Application Publication
(10) Pub. No.: US 2006/0165805 A1
(43) Pub. Date: Jul. 27, 2006

(54) Magnetic pole matrices useful for tissue engineering and treatment of disease

(76) Inventors: Gustav Steinhoff, Rethwisch (DE); Kurt Steinhoff, Kleve (DE); Wenzhong Li, Rostock (DE); Nan Ma, Rostock (DE)

Correspondence Address: JORDAN AND HAMBURG LLP, 122 EAST 42ND STREET, SUITE 4000, NEW YORK, NY, 10168, US

(21) Appl. No.: 11/317,163

(22) Filed: Dec. 22, 2005

(30) Foreign Application Priority Data
Dec. 22, 2004 (EP) EP 04106870.1

(51) Int. Cl. A61K 48/00; A61N 1/39; A61K 39/395; C12N 15/87; A61K 39/40; C12N 15/85

(52) U.S. Cl. 424/489; 435/455; 435/459; 424/133.1; 435/285.3; 607/1; 977/916

(57) ABSTRACT

A magnetic pole matrix chip facilitating the grinding of magnetic particles carrying matter effective for treating a disease or promoting tissue engineering to a disease site or a tissue engineering site, respectively

### SUMMARY OF THE INVENTION

This invention relates to magnetic pole matrices and method of use thereof for tissue engineering and targeting systematic therapy for cardiovascular disease using magnetic polymer nanoparticles gene/drug (various cytokines/growth factors/synthetic chemicals) delivery.

The magnetic pole matrices used in the present invention possesses advantages, such as distributing the magnetic nanoparticles conjugated with gene/drug (various cytokines/growth factors/synthetic chemicals) locally and uniformly on the artificial surface regulated by self-organizing behavior benefited from the magnetic pole matrices, which essentially solved the blood vessel blocking problem related to systematic therapy by magnetic nanoparticles gene/drug delivery for cardiovascular disease; promoting the adhesion of the cells (stem cells/epithelial cells/endothelial cells) labeled with magnetic beads on specific location of the artificial surface, which is very important for tissue engineering.

### BACKGROUND OF THE INVENTION

Systematic Therapy for Cardiovascular Disease

Targeting a specific area of the body is one of the main concerns associated with drug administration. Usually, large doses of the drug have to be administered to reach an acceptable therapeutic level at the desired site because only a fraction of the dose can actually reach the desired site. Further, the high dosages may also cause toxic side effects on the non-target organs (V. P. Torchilin, Drug targeting. Eur. J. Pharm. Sci. 11 Suppl. 2 (2000), pp. S81-S91). Hence targeting drug delivery to the desired site would reduce the quantity of drug required to reach local therapeutic levels at the target site, decrease the concentration of the drug at non-target sites and consequently reducing the possible side effects (V. P. Torchilin, Drug targeting. Eur. J. Pharm. Sci. 11 Suppl. 2 (2000), pp. S81-S91). Magnetic targeting drug administration incorporates magnetic particles into drug carriers, uses an externally applied magnetic field to physically direct these magnetic drug carrier particles to a desired site (V. P. Torchilin, Drug targeting Eur. J. Pharm. Sci. 11 Suppl. 2 (2000), pp. S81-S91; S. Goodwin, C. Peterson, C. Hoh and C. Bittner, Targeting and retention of magnetic targeted carriers (MTCs) enhancing intra-arterial chemotherapy. J. Magn. Magn. Mater. 194 (1999), pp. 132-139; S. Rudge, C. Peterson, C. Vessely, J. Koda, S. Stevens and L. Catterall, Adsorption and desorption of chemotherapeutic drugs from a magnetically targeted carrier (MTC). J. Control Release 74 (2001), pp. 335-340) as illustrated in FIG. 1. The magnetic particles can be injected into the bloodstream and guided to the targeted area with external magnetic fields (S. Rudge, C. Peterson, C. Vessely, J. Koda, S. Stevens, and L. Catterall, Adsorption and desorption of chemotherapeutic drugs from a magnetically targeted carrier (MTC), J. Controll. Rel., vol. 74, pp. 335-340, 2001; G. A. Flores, In-vitro blockage of a simulated vascular system using magnetorheological fluids as a cancer therapy, Eur. Cells Mater., vol. 3, pp. 9-11, 2002). Further, the magnetic particles in the magnetic fluid can interact strongly with each other, which facilitates the delivery of high concentrations of drug to desired areas. Moreover, magnetic particles composed of magnetite are well tolerated by the human body (V. P. Torchilin, Drug targeting, Eur. J. Pharm. Sci. 11 Suppl. 2 (2000), pp. S81-S91). Also, magnetic fields are not screened by biological fluids and do not interfere with most biological processes, hence they are well suited for biological applications. However, there are still several problems associated with magnetic targeting in humans which limits its application. The first limitation is associated with the influence of blood flow rate at the target site on the accumulation of magnetic particles. Therefore, much stronger magnetic fields would be required to retain magnetic particles in large arteries. Another problem associated with magnetic drug targeting in humans is the depth of the target site. Sites that are more than 2 cm deep in the body are difficult to target because the strength of the magnetic field decreases with distance (S. R. Rudge, T. L. Kurtz, C. R. Vessely, L. G. Catterall and D. L. Williamson, Preparation, characterization, and performance of magnetic iron-carbon composite microparticles for chemotherapy, Biomaterials 21 (2000), pp. 1411-1420). Moreover, although the strong interaction with each other of magnetic nanoparticles may facilitate the delivery of high concentrations of drug to targeted areas, it may also aggregate into a blot, hence blocking the blood flowing in the vessel (H. Schewe, M. Takayasu, and F. J. Friendlaender, Observation of particle trajectories in an HGMS single-wire system, IEEE Trans. Magn., vol. MAG-16, pp. 149-154, January 1980; F. J. Friedlaender, R. Gerber, W. Kurzl, and R. R. Birss, Particle motion near and capture on single spheres in HGMS, IEEE Trans. Magn., vol. MAG-17, pp. 2801-2803, November 1981; F. J. Friedlaender, R. Gerber, H. P. Henkel, and R. R. Birss, Particle buildup on single spheres in HGMS, IEEE Trans. Magn., vol. MAG-17, pp. 2804-2806, November 1981) as shown in FIG. 2.

Tissue Engineering

Endothelial seeding on biomedical devices such as artificial heart valve, stent and vessel bypass grafts plays a important role in overcoming the risk of acute thrombosis and chronic instability of the implant surface (M. Reyes, T. Lund, T. Lenvik, D. Aguiar, L. Koodie and C. M. Verfaillie, Purification and ex vivo expansion of postnatal human marrow mesodermal progenitor cells. Blood 98 9 (2001), pp. 2615-2625; S. Kaushal, G. E. Amiel, K. J. Guleserian et al., Functional small-diameter neovessels created using endothelial progenitor cells expanded ex vivo. Nat Med 7 9 (2001), pp. 1035-1040). The aims of this surface modification technique are to produce a confluent and biologically active surface with viable endothelial cells. Substantial efforts have been paid for in vitro engineering of endothelialized implants (E. L. Dvorin, J. Wylie-Sears, S. Kaushal, D. P. Martin and J. Bischoff, Quantitative evaluation of endothelial progenitors and cardiac valve endothelial cells: proliferation and differentiation on poly-glycolic acid/poly-4-hydroxybutyrate scaffold in response to vascular endothelial growth factor and transforming growth factor beta 1. Tissue Eng 9 3 (2003), pp. 487-493; Y. Zhao, D. Glesne and E. Huberman, A human peripheral blood monocyte-derived subset acts as pluripotent stem cells. Proc Natl Acad Sci USA 100 5 (2003), pp. 2426-2431). However, the lengthy preparation time to harvest, expand and culture the patient's autologous cells, and the possible cell culture contamination greatly limit the application of in vitro endothelial seeding for biomedical devices. In vivo endothelial seeding through the recruitment of circulating magnetically modified target cells to the surface of biomedical devices capable of forming a magnetic interaction with target cells could effectively solve the problems associated with the in vitro endothelial seeding WO 03/037400 A2. However, the magnetic field from the surface of devices may also cause the magnetically modified target cells aggregate into a blot on the surface of the device, which would form thrombosis in the implant surface and this may even block the blood flowing in the vessel as shown in FIG. 2. Consequently, the normal physiological function of organs dependent on those vessels may be disturbed, or even may be caused failure of the organs.

The present invention provides an economical and effective method to solve above mentioned problems by magnetic pole matrices. This allows magnetic polymer nanoparticles gene/drug (various cytokines/growth factors/synthetic chemicals) delivery and the in vivo endothelial seeding for the biomedical device being utilized in tissue engineering and systematic therapy for cardiovascular disease.

The invention provides an effective method for local targeting magnetic polymer nanoparticles for gene/drug (various cytokines/growth factors/synthetic chemicals) delivery with wanted magnetic and biological properties in relation to tissue engineering and systematic therapy for cardiovascular disease using magnetic pole matrices.

In one aspect, the invention therefore provides a process of using magnetic pole matrices as tools to manipulate the magnetic nanoparticles for gene/drug delivery. It provides a more flexible local targeting strategy used in magnetic nanoparticles for gene/drug delivery. Employment of the magnetic pole matrices in the invention has several advantages, including providing a source of strong localized magnetic field gradients at defined locations in the body for targeted drug delivery, distributing the magnetic nanoparticles on the artificial surface locally and uniformly due to the self-organizing behavior regulated by magnetic pole matrices, effectively solving the problem related to blood flow blocking due to the aggregation of magnetic nanoparticles in the vicinity of external magnet. The magnetic pole matrices suitable for the present invention are easily available and relatively inexpensive due to the large scale fabrication ability of mature VLSI (Very Large Scale Integration), ULSI (Ultra Large Scale Integration) and MEMS (Micro-Electro-Mechanical Systems) technology. The magnetic pole matrices can be used not only in systematic therapy in cardiovascular diseases, but also in tissue engineering, such as growing cells on artificial surface in vitro and in vivo.

In other aspects the invention provides methods of controlling the drug/gene dosage administered by the magnetic nanoparticles. On the one hand, the aggregation of magnetic nanoparticles in the vicinity of external block magnet may cause over high dosages delivery, which can cause toxic side effects at the target organs. On the other hand, because it is difficult for an external magnet to produce a strong and localized magnetic field, external block magnet may trap almost all of the magnetic nanoparticles in a non-target or part of target area, hence limiting its application in directing magnetic nanoparticles to delivery drug/gene to the desired area. Use of magnetic pole matrices in the process of delivering can apparently reduce the non-target aggregation of the magnetic nanoparticles and increase the ability to manipulate the drug/gene delivered uniformly, hence control the drug/gene dosage delivered by the magnetic nanoparticles.

Other novel aspects, features and advantages of the invention will become apparent to those of ordinary skill in the art up review of the following description of specific embodiments of the invention in conjunction with the accompanying figures.

### DESCRIPTION OF THE DRAWINGS

In the figures, which illustrate exemplary embodiments of the invention,

FIG. 1 is a model for trapping of circulating magnetic bead/drug/DNA complexes by external magnet;

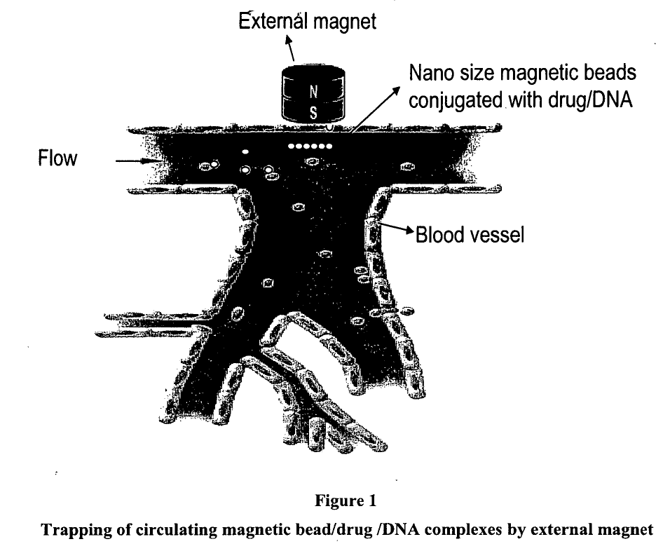

FIG. 2 shows circulating magnetic beads trapped by external magnet forming block in the blood vessel;

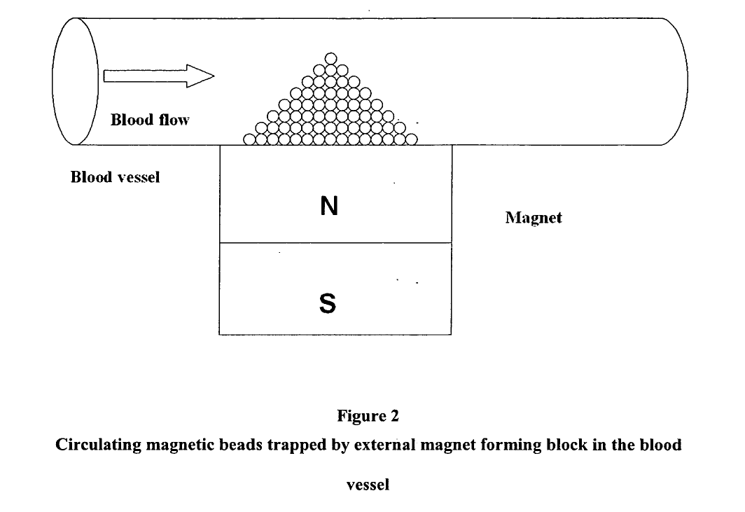

FIG. 3 shows principle for magnetic pole matrix;

FIG. 4 shows magnetic pole matrix uniformly distributes the circulating magnetic beads;

FIG. 5 is scanning electron micrograph of magnetic pole matrices with pillars heights of 100 nm and widths of 50 nm;

FIG. 6 shows PEI-magnetic beads transfection efficiency to HEK293 checked by luciferase;

FIG. 7 shows PEI-magnetic beads transfection efficiency to NIH3T3 checked by luciferase;

FIG. 8 shows PEI-magnetic beads transfection efficiency to COS7 checked by luciferase;

FIG. 9 shows PEI-magnetic beads transfection efficiency to PT67 checked by luciferase;

FIG. 10 shows gene delivery to HEK-293 (200×) by magnetic nano-particles;

FIG. 11 shows transfection specificity of magnetically controlled gene delivery;

FIG. 12 shows magnetic beads tracking of magnetically controlled gene delivery;

FIG. 13 shows gene delivery to leg muscle in mouse model by magnetic nano-particles;

FIG. 14 shows magnetic beads tracking in mouse model by tail vein injection;

FIG. 15 shows magnetic beads tracking in mouse model by tail vein injection;

FIG. 16 shows magnetic beads tracking in mouse model by tail vein injection;

FIG. 17 shows gene expression in each organ by magnetic beads delivery in mouse model via tail vein injection; and

FIG. 18 shows magnetic beads deliver therapeutic genes to the heart by tail vein injection.

### DETAILED DESCRIPTION OF THE INVENTION

The present invention provides an easy and effective process for uniformly distributing magnetic nanoparticles using magnetic pole matrices. The patterned magnetic pole matrices with feature size in nano range exhibit desired magnetic properties for magnetic polymer nanoparticles gene/drug (various cytokines/growth factors/synthetic chemicals) delivery being utilized in tissue engineering and systematic therapy for cardiovascular disease. The magnetic pole matrices of the present invention can be advantageously used for systematic therapy for cardiovascular disease and on endothelial seeding for biomedical devices so that the blocking caused by the aggregation of magnetic nanoparticles or the magnetically attracted targeting cells can be effectively eliminated.

Fabrication of Magnetic Pole Matrices

The magnetic pole matrices can be prepared by any suitable method known to a person skilled in the art and preferably by the MEMS and IT (Integrated Technology) related technology with the potential for a large scale manufacture. Electron-beam lithography enables fabrication of nano structures as small as 15 nm wide magnetic bars. X-ray lithography, imprint lithography and interferometric lithography are also available to pattern larger area samples with deep submicron feature size. Interferometric lithography could be applied to make square, rectangular, or oblique periodic arrays of circular or elongated particles, and it can cover areas of 10 cm diameter or greater in a rapid, economical process that does not require a mask. Self-assembled lithography methods, such as the use of anodized alumina or block-copolymer templates, also can be used to nanopattern large areas. Magnetic arrays were made using additive or subtractive processes, which is typical in MEMS and IT related technology. In this invention, additive processes include the deposition of magnetic material into a template, using either electrodeposition or evaporation and liftoff. In a subtractive process, the magnetic film or multilayer is deposited first and then etched using wet or dry etching methods. Aperiodic features such as servo patterns to assist dynamic control of the magnetic fields and the bond pads for electrical connections can be superposed using an additional lithography step.

Application of Magnetic Pole Matrices in Systemtic Therapy for Cardiovascular Disease

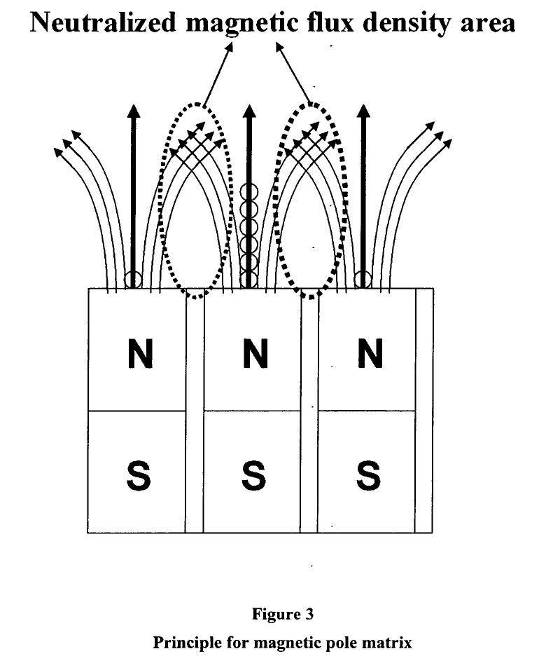

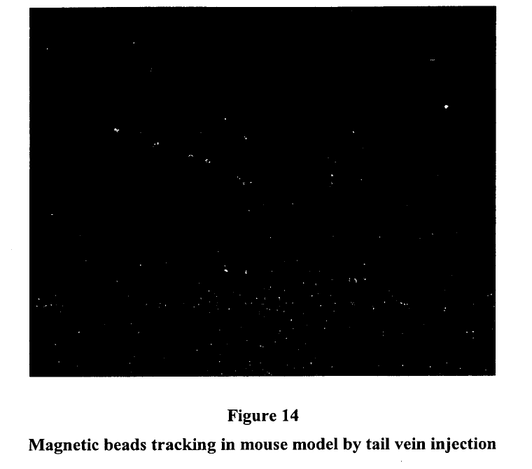

Although magnetic targeting drug administration shares many advantages over other delivery methods, the magnetic nanoparticles may also aggregate into blots, blocking the blood flow in the vessels shown as FIG. 14. Consequently, the normal physiological function of organs dependent on those vessels may be disturbed, or even cause the failure of the organs may be caused. To effectively solve the problems associated with the magnetic particles aggregation, the magnetic pole matrices were employed in this invention. The principle of magnetic pole matrix was illustrated in FIG. 3. As we know, magnetic field is the space around the magnet where its magnetic power or influence can be detected. The magnetic field is filled with magnetic lines of force. Magnetic line of force is the closed continuous curve in a magnetic field along which the north pole will move if free to do so, and its direction is given by the direction in which the isolated north pole will point. Magnetic lines of force have the following main characteristic features.

They are closed continuous curves.

They never intersect each other.

They mutually repel each other.

They contract laterally, i.e., they bend along the length of the magnet.

Outside the magnet, they travel from north to south.

Inside the magnet, they travel from south to north.

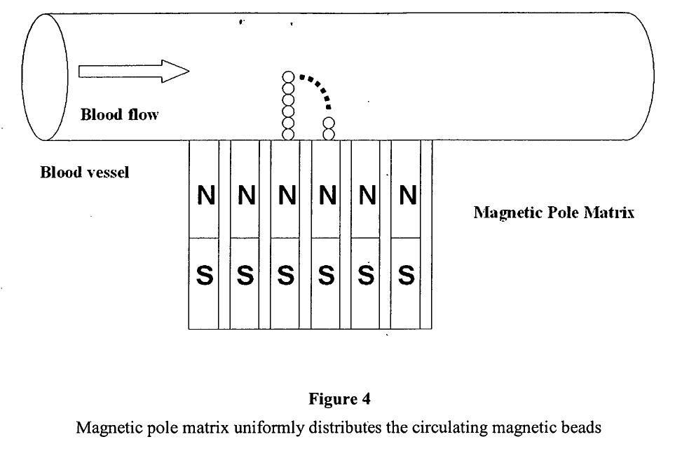

Based on the characteristics of the magnetic lines of force, we arranged the magnetic poles in regular, repetitive pattern with equal distances between neighboring units to form the magnetic pole matrices. Hence, between each two poles, there forms a neutralized magnetic flux density area. When a magnetic particle falls to a location between two magnetic poles, the magnetic particle will be attracted to either the pole in its left side or the other pole in its right side as the position between two poles is not a stable balance position of magnetic particles. Further, each magnetic pole can not attract the magnetic particles without limitation, as with the accumulation of magnetic particles in the pole direction, the magnetic particle on the top position is in a non-stable balance position and a small disturbance can make it drop to another position until it goes to a stable balance position as shown in FIG. 4. In this way, the magnetic pole matrices can automatically distribute the magnetic particles uniformly on the top area of magnetic pole matrices. Thus, the magnetic pole matrix chips integrated with electromagnetic coils would not only provide a strong local magnetic field near the targeting organ but also distribute the magnetic particles uniformly in the desired zone. And more importantly, it effectively solves the problems associated with the aggregation of the magnetic particles and provides a controllable way for the magnetic targeting systematic drug administration.

Application of Magnetic Pole Matrices in Tissue Engineering

Although in vivo magnetic endothelial seeding owns its unique potentials over other endothelial seeding methods, its application was greatly reduced due to the aggregation of magnetically modified target cells on the surface of the device, which forms the thrombosis in the implant surface. This may even block the blood flowing in the vessel. As a consequence, the normal physiological function of organs dependent on those vessels may be disturbed, or even failure of the organs may be caused. Similarly, the magnetic pole matrices in this invention can also be employed to solve above mentioned problems associated with the aggregation of magnetically modified target cells on the surface of the device. Also the magnetic pole matrix chips integrated with electromagnetic coils will also provide a strong local magnetic field under the implants, enhancing the adhesion of targeting cells modified with magnetic particles to the surface of implants. And more importantly, it effectively solves the problems associated with the aggregation of the targeting cells modified with magnetic particles and provides a controllable way for magnetic enhanced in vivo and in vitro endothelial seeding on the surface of the medical implants.

The following examples with reference to the accompanying drawing illustrate the present invention but are not limiting as to the nature of the invention.

### EXAMPLE 1

Magnetic Pole Matrices Fabrication

A magnetic pole matrix was formed as follows:

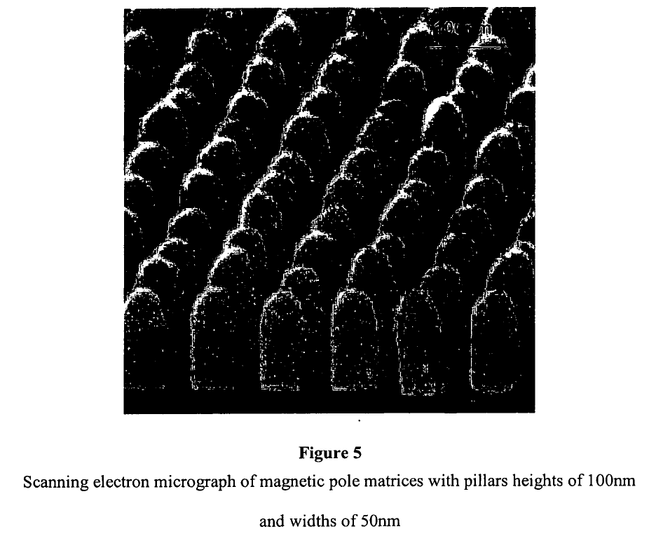

In this example, electron beam lithography and electroplating were used to produce nanoscale pillar arrays. A plating base of 10 nm Ti and 20 nm Au were evaporated on a silicon substrate. The substrate is then spin coated with polymethyl methacrylate (PMMA) positive resist of 950 kD in molecular weight. The final thickness of the PMMA was 200 nm, which determined the maximum height of the pillars. The arrays of small holes were exposed and developed in PMMA resist using electron beam lithography. The resulting structure was used as a template for the sputtering deposition of magnetic pillars. Next, magnetic arrays were made using sputtering and liftoff to deposit magnetic material into the template. In the sputtering process, magnetic film is formed over the photoresist mask. As a result, the thickness of pole matrix layer thus obtained can be determined by the sputtering rate and the sputtering time. Aperiodic features such as servo patterns to assist dynamic control of the magnetic fields and the bond pads for electrical connections, can be superposed using an additional lithography step. After the sputtering, the PMMA was removed in the acetone bath to leave the magnetic pillar arrays, shown in FIG. 5.

### EXAMPLE 2

Gene Delivery In Vitro

Gene therapy in cardiovascular system is mainly limited due to the low transfection efficiency of gene vectors in blood, in which the serum may degrade the vector's ability to deliver genes.

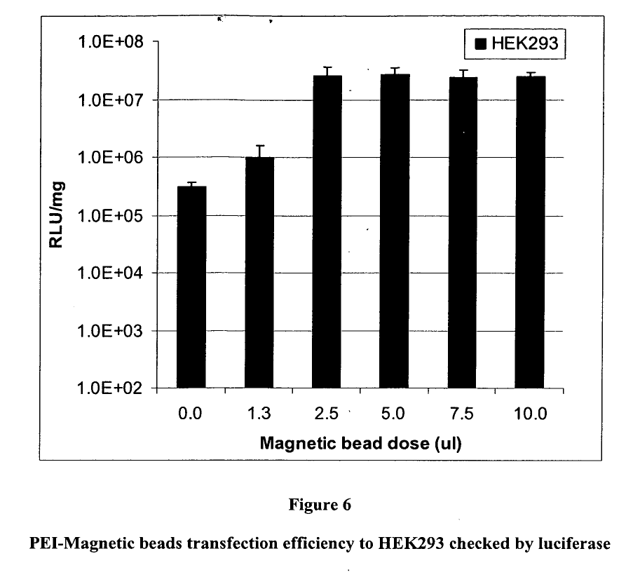

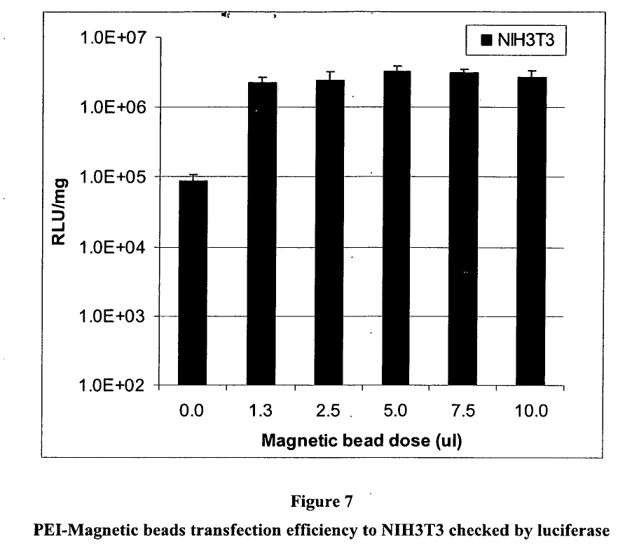

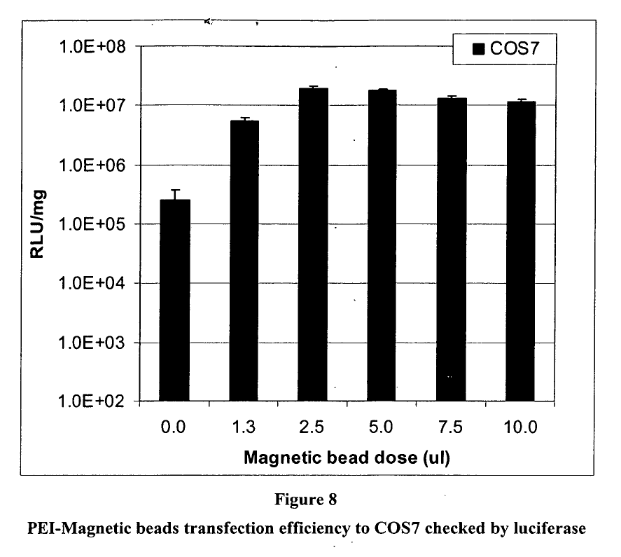

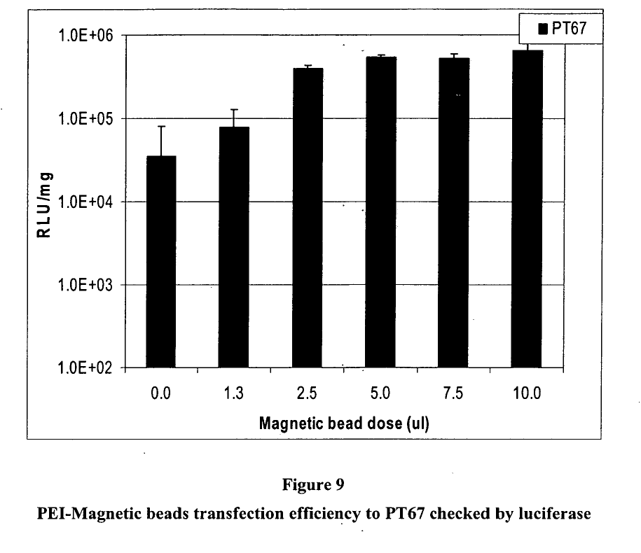

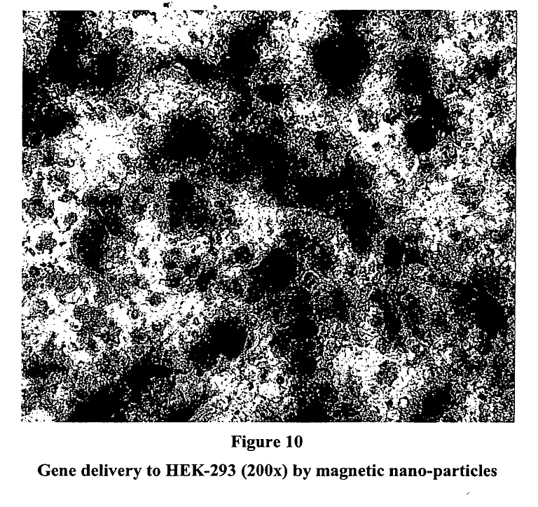

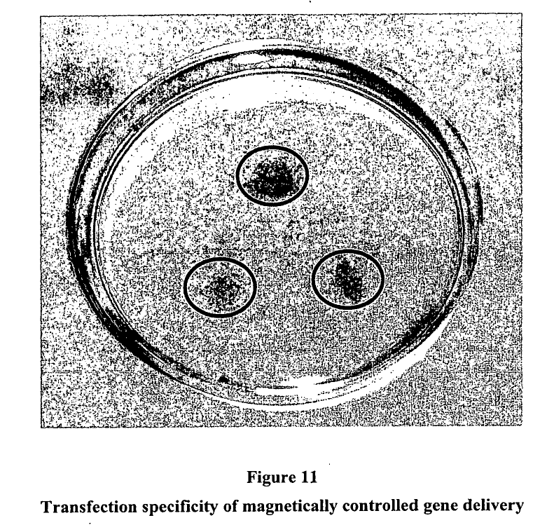

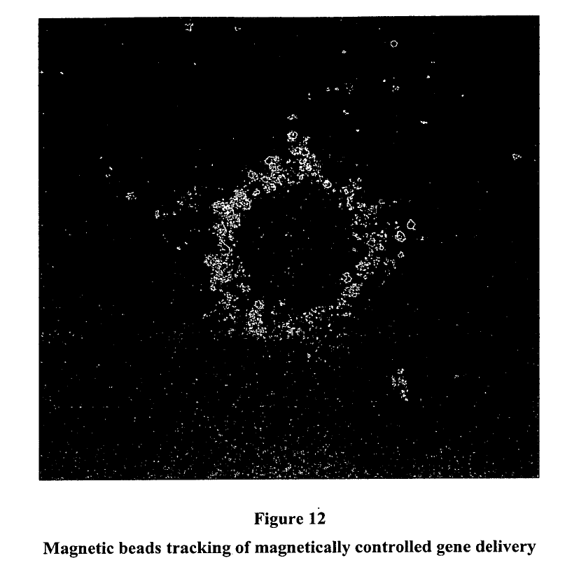

In this example, the non-viral gene vector poly-ethyleneimine (PEI) was covalently conjugated with magnetic nanobeads and desired gene by Sulfo-NHS-LC-Biotin linker to evaluate the transfection efficiency improvement. The magnetic beads/PEI/DNA complexes were found very stable even in medium with serum. It was found that magnetic beads/PEI/DNA complexes prepared in medium with serum has about 100 fold increasment of transfection efficiency than PEI/DNA complexes in 4 different cell lines tested by luciferase reporter gene as shown in FIG. 6, FIG. 7, FIG. 8 and FIG. 9. By applying three restricted external magnetic fields to 2D cell cultures, LacZ gene transfection (shown in FIG. 10) could be selectively targeted to the specific and localized cell populations as illustrated in FIG. 11. By using confocal microscopy to track the magnetic bead/PEI/DNA complex, effective endocytotic uptake and intracellular gene release with nuclear translocation were demonstrated in vitro, while the residual MNB/PEI complex localized to extranuclear lysosomes shown as in FIG. 12. Magnetic nanobeads conjugated with non-viral polymer vector provide superior transfection efficiency in vitro and in myocardium in vivo, which can be locally focused by external magnetic fields. Circumventing virus associated problems, this technique can greatly enhance the prospects of gene therapy in the cardiovascular system.

### EXAMPLE 3

Gene Delivery In Vivo

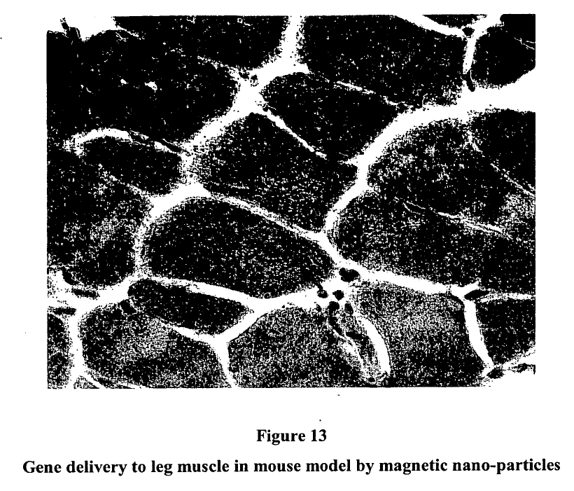

In this example, the non-viral gene vector poly-ethyleneimine (PEI) was covalently conjugated with magnetic nanobeads and reporter gene LacZ by Sulfo-NHS-LC-Biotin linker to evaluate the transfection efficacy in mouse mode. The magnetic beads/PEI/DNA complexes were prepared in medium with serum. The magnetic beads/PEI/DNA complexes with volume 50 ml were injected into the leg muscle of the mouse. LacZ gene expressions were found in the leg muscle after 72 hours injection as shown in FIG. 13. It is demonstrated that the present invention provides a feasible gene/drug delivery strategy for cardiovascular system disease.

### EXAMPLE 4

Systematic Therapy in Liver, Brain, Spleen, Heart and Kidney

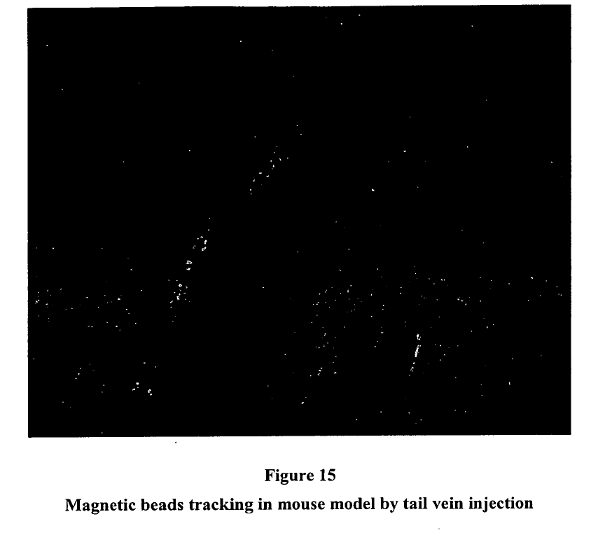

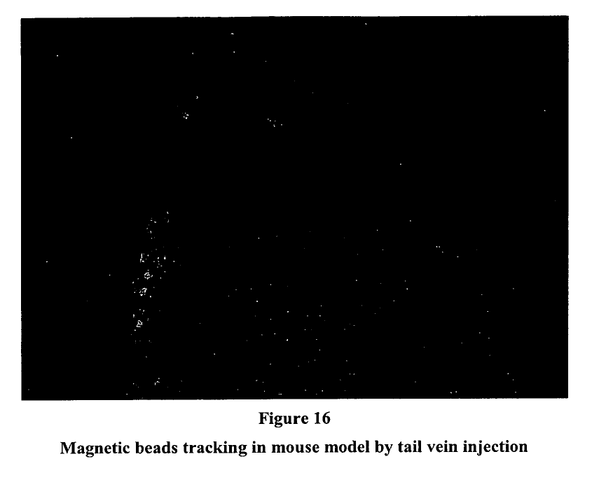

In this example, the non-viral gene vector poly-ethyleneimine (PEI) was covalently conjugated with magnetic nanobeads and fluorescent probe Oregon Green 488 by Sulfo-NHS-LC-Biotin linker to evaluate the feasibility of systematic therapy for the heart. The magnetic beads/PEI/Fluorescent Probe complexes with volume 50 ml prepared in medium with serum entered blood circulation system of mouse by the tail vein injection. The external magnet was put in the chest of the mouse for 2 hours to attract the magnetic particles circulating in the blood system. The magnetic particles were found in the heart after 72 hours injection as shown in FIG. 14, FIG. 15 and FIG. 16. It is demonstrated that the present invention provides feasible systematic therapy for cardiovascular system disease.

### EXAMPLE 5

Organ Specific Drug/Gene Delivery by Magnetic Pole Matrices

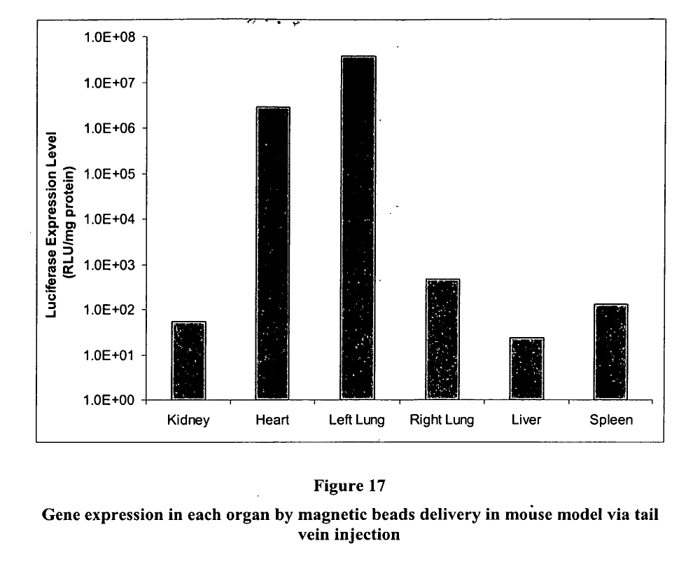

In this example, the non-viral gene vector poly-ethyleneimine (PEI) was covalently conjugated with magnetic nanobeads and luciferase gene by Sulfo-NHS-LC-Biotin linker to evaluate the gene expression in each organ. The magnetic beads/PEI/DNA complexes with volume 50 ml prepared in medium with serum entered blood circulation system of mouse by the tail vein injection. The external magnet was put in the chest of the mouse to attract the magnetic particles circulating in the blood system. The luciferase gene expressions in each organ after 72 hours injection are shown in FIG. 17. It showed that the external magnet influenced the organs have much higher gene expression than those organs without magnetic field stimulation. It is demonstrated that the present invention provides organ specific drug/gene therapy by systemic drug/gene administration.

### EXAMPLE 6

Therapeutic Gene Delivery by Magnetic Pole Matrices

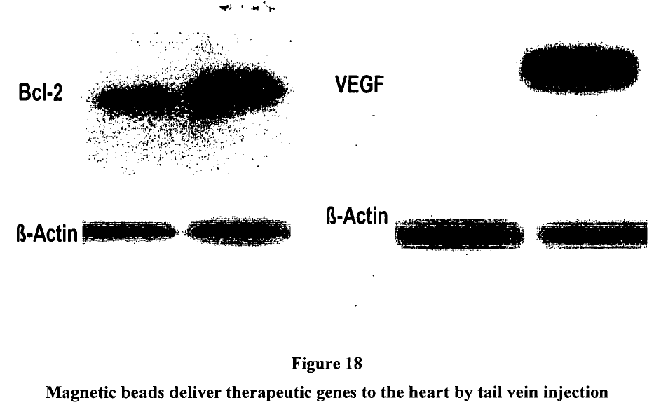

In this example, the non-viral gene vector poly-ethyleneimine (PEI) was covalently conjugated with magnetic nanobeads and therapeutic genes (Bcl-2, VEGF) by Sulfo-NHS-LC-Biotin linker to evaluate the therapeutic gene delivery to the heart. The magnetic beads/PEI/DNA complexes with volume 50 ml prepared in medium with serum entered blood circulation system of mouse by the tail vein injection. The external magnet was put in the chest of the mouse to attract the magnetic particles circulating in the blood system. The therapeutic genes were found overexpressed in the heart after 72 hours injection as shown in FIG. 18. It is demonstrated that the present invention provides feasible systematic therapeutic drug/gene therapy for cardiovascular system disease.

Other features, benefits and advantages of the present invention not expressly mentioned above can be understood form this description and the accompanying drawings by those skilled in the art.

The gene/drug delivery by magnetic nanoparticles manipulated by the magnetic pole matrices and the process for forming them described herein are all exemplary embodiments of one or more aspects of the invention. As can be understood by a person skilled in the art, many modifications to these exemplary embodiments are possible. The invention, rather, is intended to encompass all such modification within its scope, as defined by the claim.

All documents referred to herein are fully incorporated by reference.

Although the invention has been described with reference to particular embodiments, the description is only an example of the invention's application and should not be taken as a limitation. Various adaptations and combinations of features of the embodiments disclosed are within the scope of the invention as defined by the following claims.

1. (canceled)

2. The magnetic pole matrix chip of claim 35, wherein said substrate comprises silicon, and the chip further comprises a coating comprising a biocompatiable ceramic or polymer.

3. The magnetic pole matrix chip of claim 35, wherein said magnetized device comprises an electromagnetic device or a paramagnetic device.

4. The magnetic pole matrix chip of claim 3, wherein said magnetizing device is an electromagnetic device and the electromagnetic device comprises at least one magnetic core and at least one electric coil around each said magnetic core.

5. The magnetic pole matrix chip of claim 3, wherein said magnetizing device is a paramagnetic device comprising a paramagnetic or superparamagnetic material activatable by external magnetic equipment.

6. The magnetic pole matrix chip of claim 4, wherein each said magnetic core is comprised of a soft magnetic material.

7. The magnetic pole matrix chip of claim 5 in combination with said external magnetic equipment, said external magnetic equipment comprising magnetic resonance imaging (MRI) equipment or other equipment which can produce sufficiently strong magnetic field to magnetize the paramagnetic device.

8. The magnetic pole matrix chip of claim 35, wherein said magnetizable material comprises soft magnetic materials, paramagnetic materials, superparamagnetic materials, and mixtures thereof.

9. The magnetic pole matrix chip of claim 35, wherein said coating is of a polymer and the polymer is biocompatible.

10. The magnetic pole matrix chip of claim 35, wherein said bodies are of width and length in the range of 10 nm to 1 cm.

11. The magnetic pole matrix chip of claim 35, wherein said magnetizing device comprises adjustment means for adjusting magnetic field magnitude of the magnetizing device to establish a desired local magnetic field magnitude on said surface of the magnetic pole matrix.

12. The magnetic pole matrix chip of claim 35, wherein said the magnetic poles are arranged in a regular, repetitive pattern with equal distances between immediately adjacent magnetic poles thereby to establish a neutralized magnetic flux density area between each two immediately adjacent poles.

13.-34. (canceled)

35. A magnetic pole matrix chip comprising
a substrate,
a magnetizable material supported by the substrate and comprising a matrix of discrete bodies of the magnetizable material, each of the bodies being oriented with a free end thereof in the same plane as the free ends of the other of the bodies, each of the bodies being magnetizable so that the free end thereof has the same magnetic polarity as the free end of the other of the bodies, thereby to form a matrix of like magnetic poles having a planar surface, and
a magnetizing device arranged to act upon the substrate for magnetizing said bodies so that the free end of each has the same magnetic polarity.

36. A method of treating a disease or engineering tissue, comprising
introducing particles comprising a magnetic material into a patient or to an in vitro or in vivo tissue engineering cite, the particles carrying matter effective for treating the disease or contributing to formation of tissue at the tissue engineering site and the particles, and
guiding the particles to a target site comprising a site of the disease or the tissue engineering site by means of a magnetic field of the magnetic pole matrix chip of claim 35.

37. The method of claim 36, further comprising
binding the matter to the particles by conjugation thereby to produce conjugates of the matter effective for treating the disease with the particles.

38. The method of claim 37, further comprising
using chemical or biological connectors and/or spacers to facilitate preparation or use of the conjugates.

39. The method of claim 36, wherein
the target molecules are selected from the group consisting of oligonucleotides, DNA molecules, RNA molecules, proteins, antibodies, lectins and receptor molecules, or mixtures thereof.

40. The method of claim 36, further comprising
complexing the target molecules with at least one biologically active agent and/or virus by linking the at least one biologically active and/or virus to target molecules by adsorption, grafting, encapsulation or linking.

41. The method of claim 36, wherein
the particles are of size 1 nm to 1 cm.

42. The method of claim 36, further comprising
introducing the particles into the body of the patient by at least one of injection, infusion, and implantation.

43. The method of claim 36, wherein
the target site comprises an organ, implantation device, tumor, infection, aneurysms, abscess, viral growth or other focal points of disease.

44. The method of claim 36, wherein
the target cite comprises an implantation device comprised of a metal, biocompatible material, biodegradable material, bioresorbable material, polymer, ceramic and/or biological matter.

45. The method of claim 36, wherein
the target cite comprises target cells comprising stem cells, progenitor cells, endothelial cells, red blood cells, mononuclear cells, macrophages or immune system cells.

46. The method of claim 36, wherein
the target cite comprises target cells comprising autologous cells and/or donor cells.

47. The method of claim 36, wherein
the target cite comprises target cells comprising genetically manipulated cells.

48. The method of claim 36,
wherein the target cite comprises target cells, and
further comprising modifying the target cells in vivo and/or in vitro.

49. The method of claim 48, wherein
the modifying of the target cells comprises modifying surface characteristics of blood contacting surfaces of the target cells thereby to facilitate in vitro formation of cellular tissue on the blood contacting surface.

50. The method of claim 49, wherein
the cellular tissue comprises endothelial, fibrous, epithelial or bone tissue.

51. The method of claim 36,
wherein the target side comprises target cells, and
further comprising harvesting the target cells from bone marrow or fat tissue.

52. The method of claim 36,
wherein the target site comprises target cells, and further comprising culturing the target cells in vitro.

53. The method of claim 36, further comprising
introducing the target molecules or cells into the patient by means of at least one delivery vehicle.

54. The method of claim 53, wherein
the at least one delivery vehicle comprises viral vectors, liposome and polycation polymer vectors.

55. The method of claim 54, wherein
the polycation polymer vectors comprise biodegradable, biocompatible and/or bioresorbable polymers.

56. The method of claim 36, wherein
the magnetic material comprises ferromagnetic materials, ferrimagnetic material, biodegradable magnetic materials, biocompatible magnetic materials and/or bioresorbable magnetic materials.

57. The method of claim 36, wherein
biological agents, proteins and/or polymers are physically encapsulated or entrapped with the particle and/or dispersed partially or fully through the particles and/or attached or linked to the particles.

58. The method of claim 57, wherein
the polymers comprise biodegradable and/or biocompatible and/or bioresorbable polymer.
